(19) United States
(12) Patent Application Publication
(10) Pub. No.: US 2006/0204599 A1
(43) Pub. Date: Sep. 14, 2006

(54) Dietary supplement and method of using same

(76) Inventor: Jared R. Wheat, Alpharetta, GA (US)

Correspondence Address: LANIER FORD SHAVER & PAYNE, P O BOX 2087, HUNTSVILLE, AL, 35804, US

(21) Appl. No.: 11/373,955

(22) Filed: Mar. 13, 2006

Related U.S. Application Data
(60) Provisional application No. 60/661,677, filed on Mar. 14, 2005.

(51) Int. Cl. A61K 36/48; A61K 31/137

(52) U.S. Cl. 424/757; 514/649

(57) ABSTRACT

A dietary supplement is provided whereby the daily administration of materials derived from the Acacia plants is provided orally to humans for the purpose of producing or maintaining weight loss as well as improving a person's physical performance and increasing the person's lean muscle mass. The Acacia materials include these portions of the plant that are normally considered waste or inedible, such as leaves, bark, and roots. The materials can be administered in their natural form or as extracts, and can be administered in various ways including capsules and tablets. The Acacia material may also be used as a tea. For weight loss and weight control, the materials may also be administered concurrently with caloric restriction or in the absence of caloric restriction. The materials may also be administered for the purpose of increasing muscle mass concurrently with a high protein diet as well as an exercise program.

### REFERENCE TO RELATED APPLICATIONS

This application claims priority to Provisional Patent Application U.S. Ser. No. 60/661,677, entitled “Dietary Supplement,” and filed on Mar. 14, 2005, which is fully incorporated herein by reference.

### FIELD OF THE INVENTION

The present invention relates generally to dietary supplements. In particular, the present invention relates to the use of a combination of Acacia Rigidula, Acacia. Berlandieri, or Acacia Farnesiana extract with one or more ingredients to control body weight by promoting fat loss, suppressing appetite, and/or stimulating thermogenesis. More particularly, the invention relates to the dietary supplement for accomplishing these tasks.

### BACKGROUND OF THE INVENTION

Losing weight is very difficult for many individuals. Weight gain results when an individual's caloric intake exceeds the number of calories expended as energy. In attempting to lose weight, individuals often strive to attain a caloric deficit (i.e., decreasing caloric intake so that calories expended as energy exceed caloric intake). Generally, this results is an adaptive response of lowered basal (resting) metabolic rate and skeletal muscle loss, which can make it harder to keep the weight off once the individual has attained his desired weight goal.

One cause of the decrease in resting energy expenditure may be reduced functionality of the sympathetic nervous system (“SNS”) of the body. It has been observed that genetically obese rats exhibit low sympathetic outflow or responsiveness in various tissues. Humans genetically predisposed to weight gain already exhibit reduced SNS functioning; decreased energy expenditure further exacerbates their condition.

Physiologically, reduced SNS functioning translates into reduced adrenaline (norepinephrine and epinephrine) induced thermogenesis (i.e., reduced heat production within the body). Norepinephrine and epinephrine regulate metabolism via stimulation of beta-adrenergic receptors within the SNS. Beta-adrenergic receptors are involved in the pathways of lipolysis, glycogenolysis, and thermogenesis. Thus reducing SNS functioning impedes these pathways.

Indirect sympathomimetic compounds are adrenergic agents that potentiate the release of norepinephrine and epinephrine at pre-synaptic sites in the SNS and thereby avert SNS functioning resulting from weight loss or reduced caloric intake. In the past, indirect sympathomimetic drugs such as ephedrine have been administered to humans as slimming agents, often in combination with methylxanthines (stimulatory agents) such as caffeine or theophylline. While individuals undergoing such treatment may lose weight, treated individuals may also experience undesirable side affects such as nervousness, tachycardia, hypertension, insomnia, and dry mouth.

The sensation of hunger and fullness (satiety) are regulated by a complex array of interactions between neurochemicals in specific structures of the brain. The most important area in the control of eating behavior is the hypothalamus especially the ventromedial area known as the paraventricular nucleus. Imbedded in the ventromedial area of the hypothalamus are the “feeding center” and the “satiety center,” which are influenced by dietary substances (such as glucose) in the blood and by the neurotransmitters norepinephrine, serotonin and dopamine. These two centers tend to balance each other so that we want to eat but then feel a sense of satisfaction so that we stop eating. This balance can be thrown off by stress or emotional events and changes in certain hormone levels in the blood.

Norepinephrine is a natural “amine” that acts mostly at alpha-receptors in the brain, and directly or indirectly is believed to alter the hypothalamic centers in such a way as to reduce the desire for food. Serotonin and dopamine are also believed to alter eating behavior. Certain synthetic or naturally-occurring chemicals related to norepinephrine, called “sympathomimetic amines,” also have the effect of reducing food intake. This is the basis for the action of prescription drugs like phentermine and Meridia. Ephedrine (from Ephedra) is a natural sympathomimetic amine that causes the release of norepinephrine in the brain. This indirect effect contributes to its efficacy in weight-control products. The possible negative effects of ephedrine have led to the search of alternative natural and effective substances to control the desire to eat.

One alternative is a plant source of natural norepinephrine-like substance called phenethylamine. This sympathomimetic amine is found in the leaves of several species of the Acacia plant, especially Acacia Berlandieri, Acacia Rigidula and Acacia Farnesiana. Phenethylamine also occurs in cabbage, kale, cauliflower and chocolate. Phenethylamine acts on alpha-receptors in the brain, as do norepinephrine and certain prescription anti-obesity drugs. It is also believed to cause the release of dopamine in the pleasure sensing areas of the brain. The action of phenethylamine is rapidly terminated by enzymes in the intestinal wall and liver.

It has long been known that natural and synthetic substances may facilitate weight loss in those who are overweight or obese. Such substances as have found utility in this respect may act by a variety of mechanisms. For example, some such substances act by mimicking the effects of endogenous neurotransmitters and are capable of directly replacing these neurotransmitters in their actions on receptors. This, in turn, leads to increased activity of the cells which possess the receptors. Where the receptors concerned are normally responsive to the endogenous hormones adrenaline (epinephrine) and noradrenaline (norepinephrine), which meditate the activities of the sympathetic nervous system, such substances are termed direct-acting sympathicomimetic agents. Typical examples are the amphetamines. Other substances that produce similar effects on the SNS do so by stimulating the release of the endogenous hormones adrenaline and noradrenaline and are thus termed indirect-acting sympathicomimetic agents. Ephedrine is a typical example of an indirect-acting sympathicomimetic agent. Such substances may also be referred to as agonists.

While the formal distinction between direct-acting and indirect-acting sympathicomimetic action is clear, it is realized that many substances which act by causing sympathetic stimulation do so by both mechanisms, depending on intake levels and the receptors involved. Thus, amphetamines act mainly directly, but also have some indirect actions, while ephedrine acts indirectly; but, if given in higher dosage, may also stimulate receptors directly, particularly in the brain. It has been demonstrated that the main perceived actions of sympathicomimetic agents depends both on their differing specificities for the various receptors and on the pharmacokinetic behaviors of the agents in the body.

Thus, the amphetamines, which are direct agents and readily cross the blood-brain barrier, mainly cause central nervous system stimulation, while ephedrine, and particularly pseudoephedrine, are indirect agents which do not cross blood-brain barrier so readily and thus are mainly seen to exert peripheral effects.

Another class of substance of value in assisting weight loss modulates other neurotransmitters, namely, those involved in serotoninergic systems and particularly 5-hydroxytryptamine (“5-HT” otherwise known as serotonin). These substances; of which fenfluramine and its optical isomer, dexfenfluramine, are typical; act by preventing the re-uptake of serotonin into storage granules in neurones. Levels of 5-HT in the synaptic gap thus remain elevated for longer periods, exciting receptors on responsive cells to greater activity.

Other aids to weight loss have been proposed, such as substances which prevent the absorption of nutrients from the digestive system, but the value of such approaches is minimal. Generally, the accepted substances of value in weight loss act by modulating neurotransmitter function in the central nervous system or peripherally.

Substances which modulate neurotransmitter function in the central nervous system are known to act by increasing the availability of catecholamines, in particular, noradrenaline, in certain areas of the brain, thus resulting in perceived suppression of hunger. By suppressing hunger, less food is eaten, and caloric intake is lowered. Examples of such substances include phenylpropanolamine, phentermine, and the amphetamines. Substances which act by increasing the availability of 5-HT, on the other hand, are known to increase perceptions of satiety. An example of such substance is xfenfluramine.

Irrespective of mechanisms, substances of either of these types result in reduced food intake. But their use can be attended by various unwanted effects characteristic of interference with other hormone-regulated systems in the body. It has furthermore been noted that the effects of these types of substances are transient, requiring progressively greater dosage to elicit desired effects until the body finally becomes unresponsive. This progressive decrease in sensitivity is termed tachyphylaxis.

More recently, attention has been focused in ephedrine, which was originally thought to suppress the hunger center in the brain. However, during the last 30 years, research has shown that ephedrine acts mainly by stimulating thermogenesis, increasing the metabolic rate, and stimulating lipolysis.

From the foregoing, it will be obvious to those skilled in the art that the agents most suitable for inducing weight loss in those with excess weight, or, for persons of normal weight, increasing energy availability and/or muscle mass, would be sympathicomimetic (adrenergic) agents whose mechanism of action is mainly indirect, resembling that of ephedrine, and whose pharmacokinetics favor retention of the agents in the periphery rather than passage into the brain. Agents whose profiles match these requirements would be less likely to cause central nervous system stimulation under normal conditions of use, but would still possess enough central action to suppress the hunger center. The partition in favor of peripheral tissues would result in increased levels of these agents at the sites of the beta-3-receptors, which mediate lipolysis and thermogenesis. It is also widely believed that sympathicomimetic agents possessing mainly an indirect mechanism of action would be less likely to cause unwanted side effects and less likely to result in addictive situations.

Hitherto, the only such agent which has been shown to act in the optimized ideal fashion has been ephedrine itself. Ephedrine has some drawbacks, however. It is primarily provided in pharmaceutical forms which allow quick release in the body for the alleviation of acute respiratory ailments whereas, for the purpose of inducing lipolysis and thermogenesis, a slower release is desirable. Furthermore, many of those who are overweight prefer not use agents which are presented as drugs. In addition, for a variety of health conditions, such use will often be contraindicated because of the risk of potentially hazardous side effects, which risk could be increased because of the weight problem.

Prior to this invention, those wishing to avail themselves of natural products for eliciting weight loss or increasing muscle mass have had no choice other than to use products containing Ephedra herb (Ephedraceae), which contains ephedrine together with related alkaloids. However, because of concerns about the use of Ephedra herb products, many do not avail themselves of this opportunity.

The provision of a natural product that acts in the ideal fashion noted above would therefore provide major benefits to those seeking to lose weight or improve their physical fitness, or both, and would be especially useful to those who prefer not to take either drug-like products or natural products containing ephedrine alkaloids.

Thus, there exists a need in the art for a safe, effective dietary supplement that promotes weight loss and maintains lean body mass, while avoiding potential negative side effects associated with other dietary supplements used to promote weight loss.

### SUMMARY OF THE INVENTION

The present disclosure is directed to a dietary supplement which promotes weight loss, suppresses appetite, and stimulates thermogenesis. This is accomplished by combining Acacia Rigidula, Acacia Berlandieri, or Acacia Farnesiana extract with one or more other ingredients designed to modify the body's metabolism.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein.

The present invention discloses the use of adrenergic amines of the group comprising phenethylamine; N-methly-β-phenethylamine; tyramine; hordenine; N,N-dimethylphenethylamine; and N,N-dimethyl-α-methylphenethylamine that are useful to assist in weight loss, adding muscle mass, and/or increasing physical performance. The present invention also discloses the discovery that useful and exploitable levels of these adrenergic amines occur in plant species of Acacia, specifically, Acacia Rigidula, Acacia Berlandieri, and Acacia Farnesiana. Useful levels of these substances occur in the parts of the plant that are not normally eaten, including the leaves, bark, and roots, and can be extracted using methods well known to those of skill in the art.

These substances stimulate beta-receptors and release norepinephrine thereby stimulating thermogenesis, increasing metabolic rate, and stimulating lipolysis. Appetite and satiety are also regulated by these substances. For the present invention, the amount of adrenergic amines needed to be effective can be as low as 2 milligrams (mg) ingested three times daily. The preferred use, however, is to administer single doses of from 8 to 18.75 mg up to three times daily, making a total daily dose of about 25 to 56.25 mg per day.

The present invention provides a composition containing an effective amount of at least one of these adrenergic amines to stimulate the addition of lean muscle mass, enhance physical performance, promote weight control, and to stimulate weight loss. The composition can be administered in a form with the plant material, such as, but not limited to, a tablet, capsule, a tea, or other pharmacologically appropriate carrier. The composition may also be administered in a form without plant material, such as, but not limited to, a tablet, capsule, or other pharmacologically appropriate carrier. The composition should contain at least one of the group of six adrenergic amines extracted from the plant material.

The Acacia material used in accordance with the invention may consist of any portion of the plant, which contains useful amounts of the agents specified in this invention. For example, leaves of Acacia Rigidula are preferred to other parts of the plant, and may show levels of phenethylamine and related alkaloids of 5,000 ppm or more, based on dry matter, while the root or bark may contain no detectable amount of phenethylamine and related alkaloids.

Though it is possible to use a variety of Acacia materials in accordance with this invention, it is more convenient to utilize Acacia materials which already exist in appropriate form and which are generally available as traditional herbs and remedies. For example, the agents present in the residues remaining after distillation of the leaves of Acacia Rigidula plant to obtain the desired phenethylamines. In this respect, various species of the genus Acacia from other geographical locations prepared in the same way are particularly useful.

The adrenergic amines are preferably combined with one or more other substances designed to modify the body's metabolism. For example, caffeine may be included to stimulate the central nervous system and increase the metabolic rate. Other such substances include, but are not limited to: citrus aurantium, an indirect sympathomimetic agent containing synephrine alkaloids; hoodia cactus extract which helps control hunger and works through appetite suppression; cassia nomame extract, a lipase inhibitor that blocks fat absorption; yohimbe extract, a direct blocker of alpha-2 receptors, which allows fat loss in the lower body; 6,7 dihydroxybergamottin, a flavanoid used to help extend the half-life of active agents; naringen, a flavanoid used to help extend the half-life of the active agents; comniphora mukul, which helps elevate the thyroid allowing the body to burn more calories; coleus forskolin, which produces cyclic AMP, enhances glucose transport, and mimics thyroid-stimulating hormone; pilocarpus jaberandi extract, a diuretic; L-5-hydroxytryptophan, a precursor to serotonin which controls the appetite and satiety; 5-methoxytryptamine, another precursor to serotonin which controls the appetite and satiety; green tea extract which stimulates brown adipose tissue thermogenesis; phenylethylamine HCL, a central nervous stimulant, which promotes elevation of the metabolic rate; and theobroma cocoa extract, a central nervous stimulant which promotes elevation of the metabolic rate.

The use of these substances and their combination with adrenergic amines will become readily apparent to those skilled in the art from the following detailed description of an embodiment of the present.

### DETAILED DESCRIPTION

In one embodiment of the invention, material of the Acacia plant species is given to humans orally, either concurrently with caloric restriction or in the absence of caloric restriction, for the purpose of controlling body weight. The Acacia material so used is selected for its content of active agents as defined above such that the total amount of Acacia material ingested provides a sufficient amount of the active agents to achieve the desired effects. In this respect, the preferred embodiment consists of a sufficient amount defined at least 1 mg of active agents per kilogram ideal body weight per dose at any one time.

Ingestion of active agents in the range of generally about 0.05 mg to generally about 2 mg per kilogram of ideal body weight per serving will be effective in accomplishing the desired goal of weight loss, though more preferred is a range of generally about 0.75 mg. to generally about 1.5 mg per kilogram of ideal body weight. Though ingestion of larger amounts of the agents will not diminish the beneficial effects.

In this context, the active agents are deemed to be any one or more of phenethylamine; N-methly-β-phenethylamine; tyramine; hordenine; N,N-dimethylphenethylamine; and N,N-dimethyl-α-methylphenethylamine whereby the sufficient amount may be used by itself, or in combination of the agents that together provide a sufficient amount. In addition to these active agents, one or more other ingredients designed to modify the body's metabolism are added.

One embodiment includes about 375 mg Citrus Aurantium standardized for about 10% Synephrine alkaloids; about 225 mg Caffeine; about 600 mg Green Tea standardized for about 50% Polyphenels and 35% Catechins; about 75 mg Cassia Nomame extract standardized for about 8% Flavan Dimers; about 75 mg Hoodia extract standardized for about 12:1 concentrated extract; about 37.5 mg Yohimbe extract standardized for about 8% Yohimbe alkaloids; about 37.5 mg Dihydroxybergamottin; about 37.5 mg Naringen; about 37.5 mg Comniphora Mukul; about 37.5 mg Coleus Forskolin; about 37.5 mg Pilocarpus Jaborandi extract standardized for about 1.5% Pilocarpine; about 15 mg L-5 Hydroxytryptophan; about 15 mg 5-Methoxytryptamine HCL; about 150 mg Acacia Rigidula extract standardized to about 25% phenethylamine alkaloids; about 75 mg Theobroma Cocoa extract standardized to about 12% Theobromine; and about 75 mg Phenylethylamine HCL.

Another embodiment includes about 200 mg green tea extract, about 75 mg acacia rigidula extract, about 25 mg theobroma cocoa extract, about 25 mg phenylethylamine HCL, about 125 mg citrus aurantium extract, about 25 mg hoodia cactus extract, about 25 mg cassia nomame extract, about 12.5 mg yohimbe extract, about 12.5 mg 6,7 dihydroxybergamottin, about 12.5 mg naringen, about 12.5 mg comniphora mukul, about 12.5 mg coleus forskohlin, about 12.5 mg pilocarpus jaberandi extract, L-5-hydroxytryptophan, 5methoxytryptamine, and about 75 mg caffeine anhydrous.

This invention may be provided in other specific forms and embodiments without departing from the essential characteristics as described herein. The embodiments described above are to be considered in all aspects as illustrative only and not restrictive in any manner.

As described above, the present invention comprises a weight loss dietary supplement. While particular embodiments of the invention have been described, it will be understood, however, that the invention is not limited thereto, since modifications may be made by those skilled in the art, particularly in light of the foregoing teachings. It is, therefore, contemplated that the claims cover any such modifications that incorporate those features or those improvements that embody the spirit and scope of the present invention.

What is claimed is:

1. A method for promoting weight loss while stimulating thermogenesis and suppressing appetite which comprises administering to a human a composition comprising:
about 0.05 milligrams to about 2 milligrams per 1 kilogram of said human's body weight of an extract of one of the group consisting of Acacia Rigidula, Acacia. Berlandieri, and Acacia Farnesiana;

2. The method of claim 1, where said composition is in an oral dosage form.

3. The method of claim 2, wherein said oral dosage form is selected from the group consisting of a chewable tablet, a quick dissolve tablet, an effervescent tablet, a hard gelatin capsule, a soft gelatin capsule, a reconstitutable powder, a suspension, an elixor, a caplet, a health bar, a liquid, a food and combinations thereof.

4. The method of claim 2, wherein said oral dosage is selected from the group consisting of immediate release, extended release, pulsed release, delayed release, timed release, variable release, controlled release and combinations thereof.

5. The method of claim 1, wherein said composition is administered once during a twenty-four hour period of time.

6. The method of claim 1, wherein said composition is administered at least twice during a twenty-four hour period of time.

7. A method for promoting weight loss while stimulating thermogenesis and suppressing appetite which comprises administering to a human a composition comprising:
about 8 milligrams to about 56.25 milligrams of an active ingredient selected from one of the group consisting of phenethylamine, N-methly-β-phenethylamine, tyramine, hordenine, N,N-dimethylphenethylamine, and N,N-dimethyl-α-methylphenethylamine;
wherein said active ingredient is extracted from one of the group consisting of Acacia Rigidula, Acacia. Berlandieri, and Acacia Farnesiana.

8. The method of claim 7, where said composition is in an oral dosage form.

9. The method of claim 8, wherein said oral dosage form is selected from the group consisting of a chewable tablet, a quick dissolve tablet, an effervescent tablet, a hard gelatin capsule, a soft gelatin capsule, a reconstitutable powder, a suspension, an elixor, a caplet, a health bar, a liquid, a food and combinations thereof.

10. The method of claim 8, wherein said oral dosage is selected from the group consisting of immediate release, extended release, pulsed release, delayed release, timed release, variable release, controlled release and combinations thereof.

11. The method of claim 7, wherein said composition is administered once during a twenty-four hour period of time.

12. The method of claim 7, wherein said composition is administered at least twice during a twenty-four hour period of time.

13. The method of claim 7, wherein said composition further comprises citrus aurantium.

14. The method of claim 7, wherein said composition further comprises hoodia cactus;

15. The method of claim 7, wherein said composition further comprises cassia nomame extract;

16. The method of claim 7, wherein said composition further comprises yohimbe extract;

17. The method of claim 7, wherein said composition further comprises 6,7 dihydroxybergamottin,

18. The method of claim 7, wherein said composition further comprises naringen,

19. The method of claim 7, wherein said composition further comprises comniphora mukul;

20. The method of claim 7, wherein said composition further comprises coleus forskolin

21. The method of claim 7, wherein said composition further comprises pilocarpus jaberandi extract;

22. The method of claim 7, wherein said composition further comprises 5-methoxytryptamine;

23. The method of claim 7, wherein said composition further comprises green tea extract;

24. The method of claim 7, wherein said composition further comprises phenylethylamine HCL

25. The method of claim 7, wherein said composition further comprises theobroma cocoa extract.

26. A method of promoting weight loss, comprising the daily administration to a human of about 375 mg Citrus Aurantium standardized for about 10% Synephrine alkaloids; about 225 mg Caffeine; about 600 mg Green Tea standardized for about 50% Polyphenels and 35% Catechins; about 75 mg Cassia Nomame extract standardized for about 8% Flavan Dimers; about 75 mg Hoodia extract standardized for about 12:1 concentrated extract; about 37.5 mg Yohimbe extract standardized for about 8% Yohimbe alkaloids; about 37.5 mg Dihydroxybergamottin; about 37.5 mg Naringen; about 37.5 mg Comniphora Mukul; about 37.5 mg Coleus Forskolin; about 37.5 mg Pilocarpus Jaborandi extract standardized for about 1.5% Pilocarpine; about 15 mg L-5 Hydroxytryptophan; about 15 mg 5-Methoxytryptamine HCL; about 150 mg Acacia Rigidula extract standardized to about 25% phenethylamine alkaloids; about 75 mg Theobroma Cocoa extract standardized to about 12% Theobromine; and about 75 mg Phenylethylamine HCL.

27. A dietary supplement comprising about 375 mg Citrus Aurantium standardized for about 10% Synephrine alkaloids; about 225 mg Caffeine; about 600 mg Green Tea standardized for about 50% Polyphenels and 35% Catechins; about 75 mg Cassia Nomame extract standardized for about 8% Flavan Dimers; about 75 mg Hoodia extract standardized for about 12:1 concentrated extract; about 37.5 mg Yohimbe extract standardized for about 8% Yohimbe alkaloids; about 37.5 mg Dihydroxybergamottin; about 37.5 mg Naringen; about 37.5 mg Comniphora Mukul; about 37.5 mg Coleus Forskolin; about 37.5 mg Pilocarpus Jaborandi extract standardized for about 1.5% Pilocarpine; about 15 mg L-5 Hydroxytryptophan; about 15 mg 5-Methoxytryptamine HCL; about 150 mg Acacia Rigidula extract standardized to about 25% phenethylamine alkaloids; about 75 mg Theobroma Cocoa extract standardized to about 12% Theobromine; and about 75 mg Phenylethylamine HCL.

28. A dietary supplement comprising about 200 mg green tea extract, about 75 mg acacia rigidula extract, about 25 mg theobroma cocoa extract, about 25 mg phenylethylamine HCL, about 125 mg citrus aurantium extract, about 25 mg hoodia cactus extract, about 25 mg cassia nomame extract, about 12.5 mg yohimbe extract, about 12.5 mg 6,7 dihydroxybergamottin, about 12.5 mg naringen, about 12.5 mg comniphora mukul, about 12.5 mg coleus forskohlin, about 12.5 mg pilocarpus jaberandi extract, L-5-hydroxytryptophan, 5methoxytryptamine, and about 75 mg caffeine anhydrous.
